(19) United States
(12) Patent Application Publication
(10) Pub. No.: US 2025/0113985 A1
(43) Pub. Date: Apr. 10, 2025

(54) DENTAL SURGICAL FLAP RETRACTOR

(71) Applicant: KING FAISAL UNIVERSITY, Al-Ahsa (SA)

(72) Inventors: M. NAZARGI MAHABOB, AL-AHSA (SA); N. MOHAMAD AARIZ, AL-AHSA (SA)

(21) Appl. No.: 18/378,326

(22) Filed: Oct. 10, 2023

(51) Int. Cl. A61B 1/24; A61B 1/04; A61B 1/06; A61B 1/32; A61C 17/08

(52) U.S. Cl. CPC A61B 1/24; A61B 1/04; A61B 1/06; A61B 1/32; A61C 17/08

(57) ABSTRACT

A dental surgical retractor includes a first handle and a second handle. A first retractor arm extends from the first handle and has a curved retractor plate. A second retractor arm extends from the second handle and has wave-shaped elevations and depressions to adapt to the shape of a tooth. An LED light and a camera are located between the first retractor arm and the second retractor arm to illuminate and record. The first retractor arm and the second retractor arm open when the first handle and the second handle are moved toward one another.

### BACKGROUND

### 1. Field

The present disclosure relates to surgical devices, and particularly to dental surgical devices such as retractors.

### 2. Description of the Related Art

Retractors are used in oral surgery or dental surgery and are primarily involved in improving both visibility and access to the patient's mouth. They can help provide a pathway for removing foreign bodies or teeth from the oral cavity.

However, there is still a need within the dental industry for retractors to aid in the surgical extraction of impacted teeth, such as impacted mandible third molars.

### SUMMARY

A dental surgical retractor, in one embodiment, includes a first handle and a second handle. A first retractor arm extends from the first handle and has a curved retractor plate. A second retractor arm extends from the second handle and has wave-shaped elevations and depressions to adapt to the shape of a tooth. An LED light and a camera are located between the first retractor arm and the second retractor arm to illuminate and record what is being done in the oral cavity. The first retractor arm and the second retractor arm open when the first handle and the second handle are moved toward one another.

The dental surgical retractor further includes a spring located between the first handle and the second handle, spreading the first handle and second handle apart, thereby bringing the first retractor arm and the second retractor arm closer together. A coil is used instead of a spring in some embodiments.

The camera can further include a remote communication module, such as Bluetooth® wireless communication. A remote monitor that receives image information from the camera through the remote communication module is also included in some embodiments. Remote smart virtual reality (VR) eyeglasses that receive image information from the camera through the remote communication module are included in other embodiments.

The second retractor arm can include a holder that secures a suction tip. The holder can further secure a coolant supply. In other embodiments, the first retractor arm includes a holder that secures a suction tip and/or a coolant supply.

The curved retractor plate secures a flap in some embodiments.

The dental surgical retractor can further include a locking mechanism located between the first handle and the second handle. The locking mechanism is configured to keep the first handle at a fixed distance from the second handle, thereby keeping the first retractor arm at a fixed distance from the second retractor arm.

These and other features of the present subject matter will become readily apparent upon further review of the following specification.

### BRIEF DESCRIPTION OF THE DRAWINGS

FIG. 1 is an illustration of a dental surgical retractor in an open position.

FIG. 2 is an illustration of a remote monitor.

FIG. 3 is an illustration of remote smart VR eyeglasses.

FIG. 4 is an illustration of the dental surgical retractor in a closed position

Similar reference characters denote corresponding features consistently throughout the attached drawings.

### DETAILED DESCRIPTION OF THE PREFERRED EMBODIMENTS

There is need for retractors used in oral surgery or dental surgery. The needed retractors are primarily involved in moving mucoperiosteal flaps away and holding them apart from the surgical site to improve both visibility and access. Such retractors can help provide a pathway for removing foreign bodies or teeth from the oral cavity.

A self-retaining retractor permits hands-free operation during surgery. A specific self-retaining retractor for the surgical extraction of impacted mandibular third molars is needed.

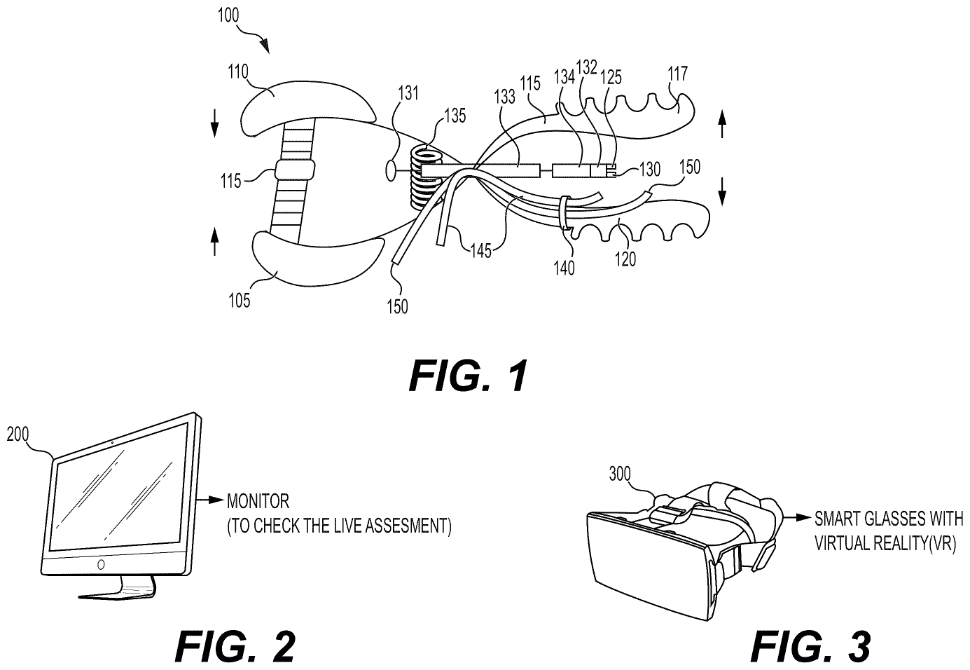

FIG. 1 is an illustration of a dental surgical retractor 100 having a first handle 105 and a second handle 110. A first retractor arm 115 extends from the first handle 105 and has a curved retractor plate 117. A second retractor arm 120 extends from the second handle 110 and has wave-shaped elevations and depressions to adapt to the shape of a tooth. An LED light 125 and a camera 130 are located between the first retractor arm 115 and the second retractor arm 120 to illuminate and record the activity taking place in the oral cavity. The first retractor arm 115 and the second retractor arm 120 open when the first handle 105 and the second handle 110 are moved toward one another.

The dental surgical retractor further includes a spring 135 located between the first handle 105 and the second handle 110. The spring 135 spreads or biases the first handle 105 and second handle 110 apart, thereby bringing the first retractor arm 115 and the second retractor arm 120 closer together. A coil can be used instead of the spring 135 in some embodiments. The coil or spring 135 produces an inverse function between the first and second handles 105,110 and the first and second retractor arms 115,120.

The camera 130 can further include a remote communication module 132, such as Bluetooth® wireless communication. A remote monitor 200, as illustrated in FIG. 2, receives image information from the camera 130 through the remote communication module 132. Remote smart VR eyeglasses 300, as illustrated in FIG. 3, receive image information from the camera 130 through the remote communication module 132. The LED light 125 provides proper illumination and live vision through the monitor 200 or the VR eyeglasses 300. The information received from the camera 130 can be further used and analyzed within a VR facility.

A rechargeable battery 134 supplies power to the LED light 125 and the camera 130. The camera 130, LED light 125 and battery 134 are detachable through the mount 133 allowing for cleaning and recharging. In this embodiment, a screw 131 extends through the mount 133 and connects to the battery 134. After surgery, this entire assembly can be removed for chemical sterilization by unscrewing the screw 131 from the battery 134.

The second retractor arm 120 can include a holder 140 that secures a suction tip 145. The holder 140 can further secure a coolant/irrigant supply 150. In other embodiments, the first retractor arm 115 includes a holder that secures a suction tip and/or a coolant supply. The holder 140, as illustrated, is a groove that secures the suction tip 145 and the coolant/irrigant supply 150.

The curved retractor plate 117, in some embodiments, secures a mucoperiosteal flap away from the surgical site to improve both visibility and access. This provides a pathway for removing foreign bodies or teeth from the oral cavity. The curved retractor plate 117, in one embodiment, is broad (2.5-3 cm), has round-edges, and is curved to hold the reflected flap. In a particular embodiment, the dimensions and shape of retractor plate 117 is useful in the surgical extraction of impacted mandibular third molars.

The dental surgical retractor 100 can further include a locking mechanism 155 located between the first handle 105 and the second handle 110. The locking mechanism 155 keeps the first handle 105 at a fixed distance from the second handle 110, thereby keeping the first retractor arm 115 at a fixed distance from the second retractor arm 120. Opposing teeth on the locking mechanism 155 keep the first handle 105 and the second handle 110 a fixed distance away. This self-retaining locking mechanism 155 allows for hands free operation during surgery.

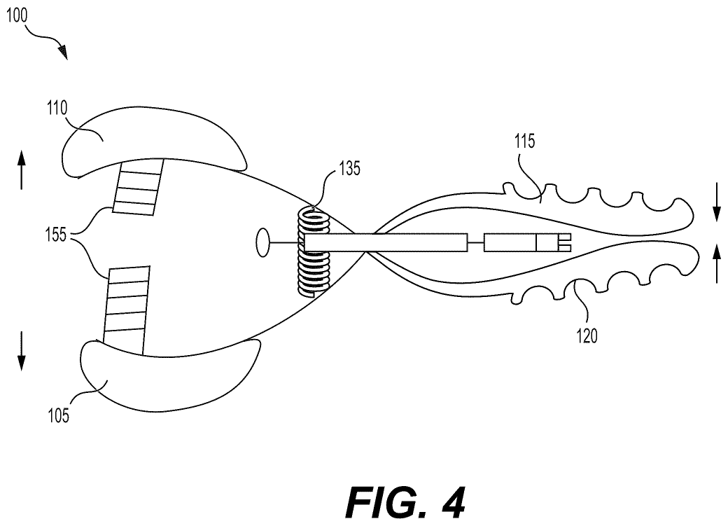

FIG. 4 is an illustration of the dental surgical retractor 100 in a closed position. In this embodiment, the spring 135 has fully expended disengaging the locking mechanism 155 and spreading the handles 105,110 apart. The inverse function forces first and second retracting arms 115,120 together into a closed position.

A self-retaining dental flap retractor for the surgical extraction of mandibular third molars is disclosed. The retractor has two arms and a handle with a coil/spring therebetween to produce an inverse function and keep the arms open. A first arm has wave-shaped elevations and depressions to adapt to the tooth for support. A second arm containins a broad (2.5-3 cm), round-edged, and curved flap retractor plate. A detachable LED light with a small camera is provided where the camera is connected via Bluetooth® wireless communication to transmit a camera image either to a proximal monitor or a smart eyeglasses/visors with VR capabilities. Both the LED light and the camera get power from detachable and rechargeable lithium batteries. On the buccal side of the first arm is a small groove to situate a suction tip and/or a coolant tube.

It is to be understood that the dental surgical retractor is not limited to the specific embodiments described above, but encompasses any and all embodiments within the scope of the generic language of the following claims enabled by the embodiments described herein, or otherwise shown in the drawings or described above in terms sufficient to enable one of ordinary skill in the art to make and use the claimed subject matter.

1. A dental surgical retractor, comprising
a first handle and a second handle;
a first retractor arm extending from the first handle and having a curved retractor plate located at a distal end of said first retractor arm;
a second retractor arm extending from the second handle and having a non-planar surface of wave-shaped elevations and depressions configured to adapt to the shape of a tooth and located at a distal end of said second retractor arm; and
an LED light and a camera, each located between the first retractor arm and the second retractor arm to illuminate and record;
wherein the first retractor arm and the second retractor arm open when the first handle and the second handle are moved toward one another,
wherein a rechargeable battery is also located between the first retractor arm and the second retractor arm and is also adjacent the LED light and the camera and provides power to the LED light and the camera while they illuminate and record, and wherein the rechargeable battery, LED light and the camera are all detachable from a mount located between the first retractor arm and the second retractor arm.

2. The dental surgical retractor as recited in claim 1, further comprising a spring located between the first handle and the second handle spreading the first handle and second handle apart thereby bringing the first retractor arm and the second retractor arm closer together.

3. The dental surgical retractor as recited in claim 1, further comprising a coil located between the first handle and the second handle spreading the first handle and second handle apart thereby bringing the first retractor arm and the second retractor arm closer together.

4. A system comprising: the dental surgical retractor as recited in claim 1, wherein the camera further comprises a remote communication module.

5. The system as recited in claim 4, wherein the remote communication module is Bluetooth®.

6. The system as recited in claim 4, further comprising a remote monitor that receives image information from the camera through the remote communication module.

7. The system as recited in claim 4, further comprising remote smart VR eyeglasses that receive image information from the camera through the remote communication module.

8. The dental surgical retractor as recited in claim 1, wherein the second retractor arm further comprises a holder that secures a suction tip.

9. The dental surgical retractor as recited in claim 8, wherein the holder further secures a coolant supply.

10. The dental surgical retractor as recited in claim 1, wherein the first retractor arm further comprises a holder that secures a suction tip.

11. The dental surgical retractor as recited in claim 10, wherein the holder further secures a coolant supply.

12. The dental surgical retractor as recited in claim 1, wherein the curved retractor plate is configured to secure a flap.

13. The dental surgical retractor as recited in claim 1, further comprising a locking mechanism located between the first handle and the second handle, the locking mechanism keeping the first handle at a fixed distance from the second handle thereby keeping the first retractor arm at a fixed distance from the second retractor arm.
